(19) United States
(12) Patent Application Publication
(10) Pub. No.: US 2008/0306285 A1
(43) Pub. Date: Dec. 11, 2008

(54) Heat-Labile Prodrugs

(75) Inventors: Ron L. Hale, Woodside, CA (US); Dennis W. Solas, San Francisco, CA (US); Kathleen Simis, San Mateo, CA (US); Amy T. Lu, Los Altos, CA (US); Peter M. Lloyd, Walnut Creek, CA (US)

Correspondence Address: SWANSON & BRATSCHUN, L.L.C, 8210 SOUTHPARK TERRACE, LITTLETON, CO, 80120, US

(73) Assignee: ALEXZA PHARMACEUTICALS, INC., Mountain View, CA (US)

(21) Appl. No.: 12/111,188

(22) Filed: Apr. 28, 2008

Related U.S. Application Data
(60) Provisional application No. 60/914,584, filed on Apr. 27, 2007.

(51) Int. Cl. C07D 311/80; C07C 39/06; C07J 1/00

(52) U.S. Cl. 549/390; 568/781; 552/625

(57) ABSTRACT

Disclosed herein are heat-labile prodrugs, their preparation and uses.

### CROSS-REFERENCES TO RELATED APPLICATIONS

This application claims priority to U.S. Provisional Application No. 60/914,584, filed on Apr. 27, 2007, the entire teachings of which are incorporated herein by reference.

### STATEMENT REGARDING FEDERALLY SPONSORED RESEARCH OR DEVELOPMENT

This invention was made with Government support under Grant No. 1 R43 CA94614-01, awarded by the National Institutes of Health. The Government has certain rights in the invention.

### FIELD OF THE INVENTION

The present invention relates to heat-labile prodrugs, their preparation and uses.

### BACKGROUND OF THE INVENTION

Pharmaceutical compounds are subject to degradation by a number of physical or chemical mechanisms, including oxidation, hydrolysis and photolysis, thereby potentially reducing efficacy, impacting safety, and limiting shelf life. Volatile pharmaceutical compounds are also subject to loss due to evaporation. In addition, some pharmaceutical compounds have physical properties that may be undesirable. For example, drugs that are liquids or resins may be difficult to formulate.

A number of drug delivery devices and methods have been described that comprise heating the drug. International application WO 94/09842 to Rosen describes a device with an electric heating element that vaporizes a predetermined amount of some agents. U.S. Pat. Nos. 4,917,119 to Potter et al.; 4,941,483 to Ridings et al.; 5,099,861 to Clearman et al.; 4,922,901 to Brooks et al.; 4,303,083 to Buruss, Jr.; 7,128,067 to Byron et al.; and 7,090,830 to Hale et al. also describe devices that vaporize various medications.

### SUMMARY OF THE INVENTION

The present invention discloses prodrugs, and salts thereof, that are converted by heating to pharmaceutical compounds. In preferred embodiments, the prodrugs are converted by heating during vaporization. In preferred embodiments, the precursor compound has improved stability during manufacture or storage, is less subject to evaporative loss, and/or exists in a preferred physical state as compared to the pharmaceutical composition.

In some embodiments, the prodrug of a phenolic drug compound has the general structural formula:

DRUG-O—(CR¹R²)ₙCOOR³

wherein DRUG-O— is a hydroxyl functional group attached to a carbon atom of an aromatic ring of the phenolic drug compound; R¹, R² and R³ are independently selected from the group consisting of H, cycloalkyl groups having up to 10 carbon atoms, straight or branched chain alkyl, alkenyl or alkynyl groups of 1 to 10 carbon atoms, wherein the chains thereof (i) may be interrupted by at least one N, S, or O atom, or (ii) may be substituted by at least one group selected from the group consisting of COR⁴, COOR⁴ and CON(R⁴)₂, hydrocarbyl aryl groups, aryl groups substituted by at least one group selected from the group consisting of COR⁴, COOR⁴, CON(R⁴)₂, N(R⁴)₂, OR⁴, halogen, SR⁴, NO₂, and R⁴, mono- bi-cyclic saturated or unsaturated heterocyclic rings, each ring consisting of 3 to 7 members selected from the group consisting of carbon, nitrogen, oxygen and sulfur, CN, COR⁴, COOR⁴, CON(R⁴)₂, and C(halogens)₃; R⁴ is selected from the group consisting of cycloalkyl groups having up to 10 carbon atoms, straight or branched chain alkyl, alkenyl and alkynyl groups having 1 to 10 carbon atoms, straight or branched chain alkyl, alkenyl and alkynyl groups of 1 to 10 carbon atoms wherein the chains thereof may be interrupted by at least on N, S or O atom, hydrocarbyl aryl groups, and in the case of —N(R⁴)₂ taken with the other R⁴ group and N is mono- or bi-cyclic saturated or unsaturated heterocyclic ring, wherein each ring consists of 3 to 7 members selected from the group consisting of carbon, nitrogen, oxygen and sulfur; and n is 1 to 3. In some preferred embodiments, R¹ is H and the phenolic drug compound is selected from the group consisting of Δ⁹-tetrahydrocannabinol, propofol, and estradiol.

In other embodiments, the prodrug of a phenolic drug compound has general structural formula:

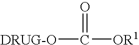

wherein DRUG-O— is a hydroxyl functional group attached to a carbon atom of an aromatic ring of the phenolic drug compound; R¹ is selected from the group consisting of H, cycloalkyl groups having up to 10 carbon atoms, straight or branched chain alkyl, alkenyl or alkynyl groups of 1 to 10 carbon atoms, wherein the chains thereof (i) may be interrupted by at least one N, S, or O atom, or (ii) may be substituted by at least one group selected from the group consisting of COR², COOR² and CON(R²)₂, hydrocarbyl aryl groups, aryl groups substituted by at least one group selected from the group consisting of COR², COOR², CON(R²)₂, N(R²)₂, OR², halogen, SR², NO₂, and R², mono- bi-cyclic saturated or unsaturated heterocyclic rings, each ring consisting of 3 to 7 members selected from the group consisting of carbon, nitrogen, oxygen and sulfur, CN, COR², COOR², CON(R²)₂, and C(halogens)₃; and R² is selected from the group consisting of cycloalkyl groups having up to 10 carbon atoms, straight or branched chain alkyl, alkenyl and alkynyl groups having 1 to 10 carbon atoms, straight or branched chain alkyl, alkenyl and alkynyl groups of 1 to 10 carbon atoms wherein the chains thereof may be interrupted by at least on N, S or O atom, hydrocarbyl arl groups, and in the case of —N(R²)₂ taken with the other R² group and N is mono- or bi-cyclic saturated or unsaturated heterocyclic ring, wherein each ring consists of 3 to 7 members selected from the group consisting of carbon, nitrogen, oxygen and sulfur. In some preferred embodiments, R¹, R² and R³ are H, n is 2, and the phenolic drug compound is selected from the group consisting of Δ⁹-tetrahydrocannabinol, propofol, and estradiol.

In other embodiments, the prodrug of a phenolic drug compound has general structural formula:

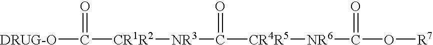

wherein DRUG-O— is a hydroxyl functional group attached to a carbon atom of an aromatic ring of the phenolic drug compound; R¹, R², R³, R⁴, R⁵, R⁶ and R⁷ are independently selected from the group consisting of H, cycloalkyl groups having up to 10 carbon atoms, straight or branched chain alkyl, alkenyl or alkynyl groups of 1 to 10 carbon atoms, wherein the chains thereof (i) may be interrupted by at least one N, S, or O atom, or (ii) may be substituted by at least one group selected from the group consisting of COR⁸, COOR⁸ and CON(R⁸)₂, hydrocarbyl aryl groups, aryl groups substituted by at least one group selected from the group consisting of COR⁸, COOR⁸, CON(R⁸)₂, N(R⁸)₂, OR⁸, halogen, SR⁸, NO₂, and R⁸, mono- bi-cyclic saturated or unsaturated heterocyclic rings, each ring consisting of 3 to 7 members selected from the group consisting of carbon, nitrogen, oxygen and sulfur, CN, COR⁸, COOR⁸, CON(R⁸)₂, and C(halogens)₃; and R⁸ is selected from the group consisting of cycloalkyl groups having up to 10 carbon atoms, straight or branched chain alkyl, alkenyl and alkynyl groups having 1 to 10 carbon atoms, straight or branched chain alkyl, alkenyl and alkynyl groups of 1 to 10 carbon atoms wherein the chains thereof may be interrupted by at least on N, S or O atom, hydrocarbyl arl groups, and in the case of —N(R⁸)₂ taken with the other R⁸ group and N is mono- or bi-cyclic saturated or unsaturated heterocyclic ring, wherein each ring consists of 3 to 7 members selected from the group consisting of carbon, nitrogen, oxygen and sulfur. In some preferred embodiments, R¹, R², R³, R⁴, R⁵, R⁶ and R⁷ are H and the phenolic drug compound is selected from the group consisting of Δ⁹-tetrahydrocannabinol, propofol, and estradiol.

In other embodiments, the prodrug of a phenolic drug compound has general structural formula:

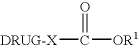

wherein DRUG-X— is a carbon atom of an aromatic ring of the phenolic drug compound in the o- or p- position relative to a hydroxyl functional group attached to a different carbon atom of said aromatic ring; R¹ selected from the group consisting of H, cycloalkyl groups having up to 10 carbon atoms, straight or branched chain alkyl groups of 1 to 10 carbon atoms. In some preferred embodiments, R¹ is H and the phenolic drug compound is selected from the group consisting of Δ⁹-tetrahydrocannabinol, propofol, and estradiol.

In some embodiment, the invention provides a method of making a phenolic drug compound comprising heating a composition comprising a prodrug of the phenolic drug compound to a temperature greater than 100°.

In some embodiments, the invention provides a method of making a vapor comprising a phenolic drug compound comprising heating a composition comprising a prodrug of the phenolic drug composition to a temperature sufficient to vaporize at least a portion of the composition to generate a vapor comprising the phenolic drug compound. In some embodiments, the vapor is condensed (e.g., by cooling) to form an aerosol.

### BRIEF DESCRIPTION OF THE DRAWINGS

Further features and advantages will become apparent from the following description of various embodiments of the invention, as illustrated in the accompanying drawings in which:

FIG. 1 illustrates a general scheme for thermal decomposition of prodrugs of the invention comprising 2-carboxyethyl derivatives of phenolic drug compositions.

FIG. 2 illustrates a general scheme for thermal decomposition of prodrugs of the invention comprising t-butoxycarbonyl derivatives of phenolic drug compositions.

FIG. 3 illustrates a general scheme for thermal decomposition of prodrugs of the invention comprising 2-carboxylic acid derivatives of phenolic drug compositions.

FIG. 4 illustrates a general scheme for thermal decomposition of prodrugs of the invention comprising t-butoxycarbonyl-glycinyl-glycinate derivatives of phenolic drug compositions.

FIG. 5 illustrates the conversion of 1-(2-carboxyethyl)-Δ⁹-THC to Δ⁹-THC, carbon dioxide, and ethylene upon heating.

FIG. 6 illustrates the conversion of 1-(t-butoxycarbonyl)-Δ⁹-THC to Δ⁹-THC, carbon dioxide, and isobutylene upon heating.

FIG. 7 illustrates the conversion of Δ⁹-THC-2-carboxylic acid (THCA) to Δ⁹-THC upon heating.

FIG. 8 illustrates the conversion of Δ⁹-THC-[1-(t-butoxycarbonyl-glycinyl-glycinate)] to Δ⁹-THC, carbon dioxide, isobutylene, and 2,5-diketopiperazine upon heating.

FIG. 9 illustrates the conversion of O-(2-carboxyethyl)-propofol to propofol, carbon dioxide, and ethylene upon heating.

FIG. 10 illustrates the conversion of O-(t-butoxycarbonyl)-propofol to propofol, carbon dioxide, and isobutylene upon heating.

FIG. 11 illustrates the conversion of 3-t-butoxycarbonyl-estradiol to estradiol

FIG. 12 illustrates the conversion of 3-(t-butoxycarbonyl-glycinyl-glycinate)-estradiol to estradiol.

FIG. 13 is a plot showing Δ⁹-THC formed as a function of THCA coated film thickness and vaporization temperatures.

FIG. 14 is a plot showing arterial plasma concentration of Δ⁹-THC and THCA in a canine model as a function of time.

FIG. 15 is a plot showing venous plasma concentration of Δ⁹-THC and THCA in a canine model as a function of time.

FIG. 16 is a bar graph showing aerosol purity of propofol as a function of vaporization temperature.

FIG. 17 is a bar graph showing percent estradiol in aerosol as a function of vaporization temperature.

FIG. 18 is a bar graph showing percent prodrug in aerosol as a function of vaporization temperature.

### DETAILED DESCRIPTION OF THE PREFERRED EMBODIMENTS

While the present invention is capable of being embodied in various forms, the description below of several embodiments is made with the understanding that the present disclosure is to be considered as an exemplification of the claimed subject matter, and is not intended to limit the appended claims to the specific embodiments illustrated.

Before the present invention is described in detail, it is to be understood that, unless otherwise indicated, this invention is not intended to be limited to specific pharmaceutically active compounds or drugs, as such may vary. It is also to be understood that the terminology used herein is for the purpose of describing particular embodiments only, and is therefore not intended to limit the scope of the present invention.

It must be noted that, as used herein and in the claims, the singular forms “a”, “and”, and “the” include plural referents, unless the context clearly dictates otherwise. Thus, for example, reference to “a prodrug” includes one or more prodrugs.

As used herein, the term “physiologically active compound” refers to a chemical compound that alters, affects, treats, cures, prevents, or diagnoses a disease after the compound is administered to a mammalian body. Physiologically active compounds may be referred to hereinafter simply as “compounds” or “drugs”.

As used herein, a “prodrug” is a compound that can be chemically converted in vitro into a physiologically active compound, i.e., it is a precursor of a desired physiologically active compound. Typically, the prodrug does not have physiological activity, but the term is not so limited and encompasses compounds that may have physiological activity. A “heat-labile” or “thermally labile” prodrug is a prodrug that can be converted into physiologically active compound through heating, i.e., subjecting the prodrug to an elevated temperature.

As used herein, a “phenolic compound” is a compound that includes at least one hydroxy functional group attached to a carbon atom of an aromatic ring. A “phenolic drug compound” is a phenolic compound that also is a pharmaceutically active compound.

There are a number of thermally reversible reactions that can be used to generate a desired pharmaceutically active compound from a suitable precursor. These include without limitation, thermally-induced decarboxylation, reverse Diels-Alder condensations, olefin elimination (N-isobutyl ammonium drugs and other Hoffman degradation reactions), other elimination reactions such as nitrogen elimination from polynitrogen compounds, rearrangements, and reverse Michael reactions. These thermally reversible reactions can be used to prepare the heat-labile prodrugs of the invention.

In some of the drug delivery devices and methods that comprise heating a drug, the drug is first deposited on a substrate. In such embodiments, the thermally reversible reactions discussed above may be used to attach the drug compound to the substrate. For example, a volatile compound may be attached to a chemically modified substrate that has been modified by coating with a nonvolatile polymer having reactive functional groups or covalently modified with a reactive group, via a covalent bond that would be broken upon heating. When the drug compound is heated, the bond between the substrate (or a polymer or other chemical moiety attached to the substrate) is broken and the drug compound is released. In a preferred embodiment, products of the reaction (other than the freed drug compound) would be retained on the substrate. This approach may be most effective for volatile drugs where the thermal reaction and vaporization can be achieved at relatively low temperatures that do not lead to unwanted thermal breakdown of the polymer or attaching group itself.

In other embodiments, the prodrugs of the invention may be deposited on a substrate, e.g., coated as a thin film, without the creation of any covalent bond between the substrate (or a polymer or other chemical moiety attached to the substrate). Upon heating, the prodrug decomposes to generate the drug and any by-products. In a preferred embodiment, the by-products are not toxic.

For use in the present invention, the prodrug is typically a solid at standard temperature and pressure.

The prodrug is typically a derivative of a phenolic drug compound. In preferred embodiments, the prodrug is selected from the group consisting of a t-butoxycarbonyl derivative of a phenolic drug compound, a carboxylic acid derivative of a phenolic drug compound, and a t-butoxycarbonyl-glycinyl-glycinate-derivative of a phenolic drug compound.

Phenolic drug compounds useful in the present invention include without limitation, Δ⁹-tetrahydrocannabinol (Δ⁹-THC), propofol, estradiol, apomorphine, dopamine, epinephrine, and related compounds.

In one preferred embodiment, the phenolic drug compound is Δ⁹-THC, and the prodrug is selected from the group consisting of 1-(t-butoxycarbonyl)-Δ⁹-THC, THC-2-carboxylic acid (THCA), and Δ⁹-THC-[1-(t-butoxycarbonyl-glycinyl-glycinate).

In another preferred embodiment, the phenolic drug compound is propofol, and the prodrug is selected from the group consisting of O-(2-carboxyethyl)-propofol and O-(t-butoxycarbonyl)-propofol.

In yet another embodiment, the drug is estradiol, and the prodrug is selected from the group consisting of 3-t-butoxycarbonyl-estradiol, estradiol-[3-(t-butoxycarbonyl-glycinyl-glycinate)], and 3-(2-carboxyethyl)-estradiol.

In a method aspect of the invention, the method comprises heating a prodrug of a phenolic drug compound to a temperature sufficient to convert at least a portion of the prodrug to the phenolic drug compound.

In another method aspect of the invention, the method comprises heating a composition comprising a prodrug of a phenolic drug compound to a temperature sufficient to vaporize at least a portion of the composition and form a vapor comprising the phenolic drug compound.

In another method aspect of the invention, the method comprises heating a composition comprising a prodrug of a phenolic drug compound to a temperature sufficient to vaporize at least a portion of the composition and form a vapor comprising the phenolic drug compound, and condensing the vapor to form an aerosol.

The precursor compound is typically heated to a temperature of at least 100° C.; more typically, the precursor compound is heated to a temperature within the range of 100° C. to 400° C.

Preferably, heating of the precursor compound produces essentially no toxic by-products.

### I. THC Prodrugs

THC (Δ⁹-tetrahydrocannabinol) is the primary active compound in marijuana (Cannabis sp.) and has garnered increasing attention in the medical community as a result of its complex and widespread systemic effects. The medical indications that have been reported for Δ⁹-THC (and other cannabinoids) are numerous and most notably include appetite stimulation in patients with AIDS, nausea and vomiting associated with chemotherapy, and neuropathic pain and spasticity associated with multiple sclerosis.

Δ⁹-THC is a moisture- and light-sensitive viscous liquid with poor shelf-life stability. Several thermally labile solid precursors of THC have been identified that meet chemical and physical shelf-stability requirements. When heated to vaporization temperatures, an amount of the precursor is converted to Δ⁹-THC (typically, about 90%) to form a vapor comprising both Δ⁹-THC and unconverted precursor. The vapor may be cooled under conditions effective to create a condensation aerosol comprising Δ⁹-THC and unconverted precursor.

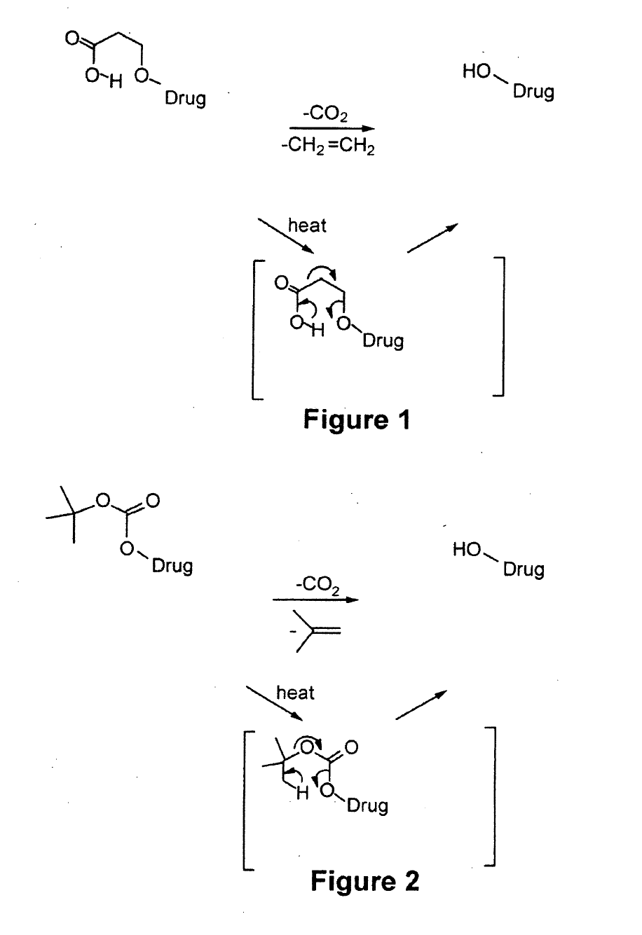

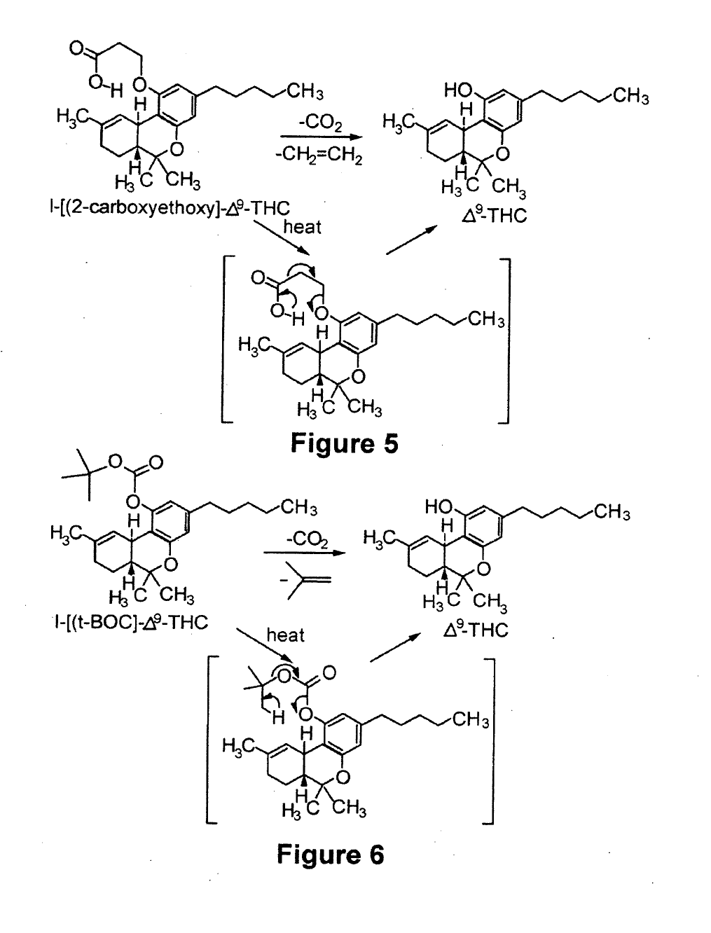

As shown in FIG. 5, a 2-carboxyethyl derivative of Δ⁹-THC (1-[2-carboxyethyoxy]-Δ⁹-THC] is thermally converted to Δ⁹-THC via a reverse Michael addition-type reaction, with carbon dioxide and ethylene as by-products. The general scheme for thermal conversion of a t-butoxycarbonyl derivative of a drug is shown in FIG. 1.

As shown in FIG. 6, a t-butoxycarbonyl derivative of Δ⁹-THC (1-[t-butoxycarbonyl]-Δ⁹-THC] is thermally converted to Δ⁹-THC, with carbon dioxide and isobutylene as by-products. The general scheme for thermal conversion of a t-butoxycarbonyl derivative of a drug is shown in FIG. 2.

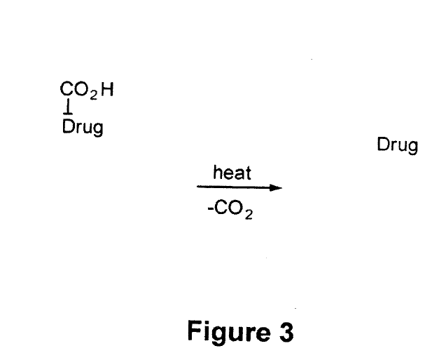

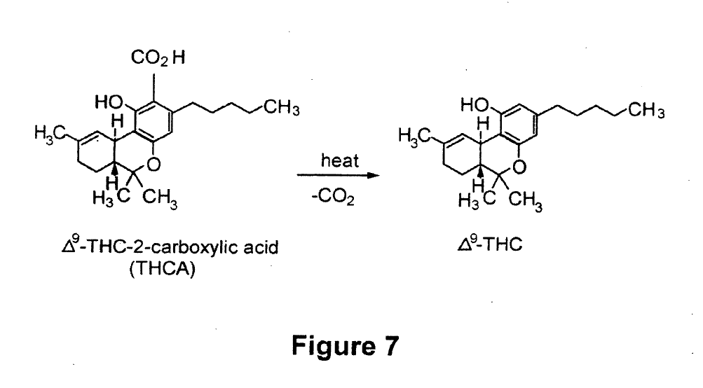

As shown in FIG. 7, a 2-carboxylic acid derivative of Δ⁹-THC (Δ⁹-THC-2-carboxylic acid) is thermally converted to Δ⁹-THC via a decarboxylation reaction, with carbon dioxide as a by-product. The 4-carboxylic acid derivative of Δ⁹-THC (Δ⁹-THC-4-carboxylic acid) undergoes a similar decarboxylation reaction to produce Δ⁹-THC and the by-product carbon dioxide. The general scheme for thermal conversion of a carboxylic acid derivative of a drug is shown in FIG. 3.

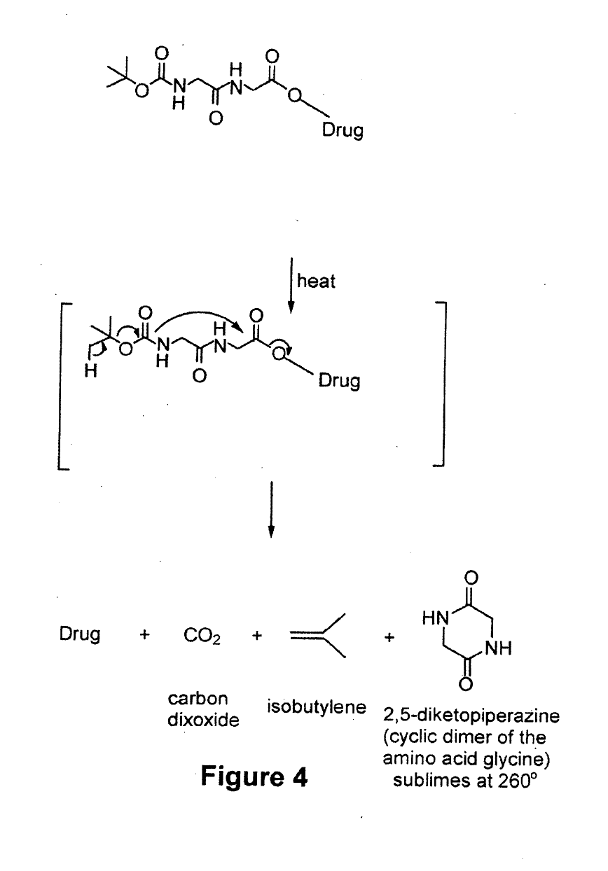

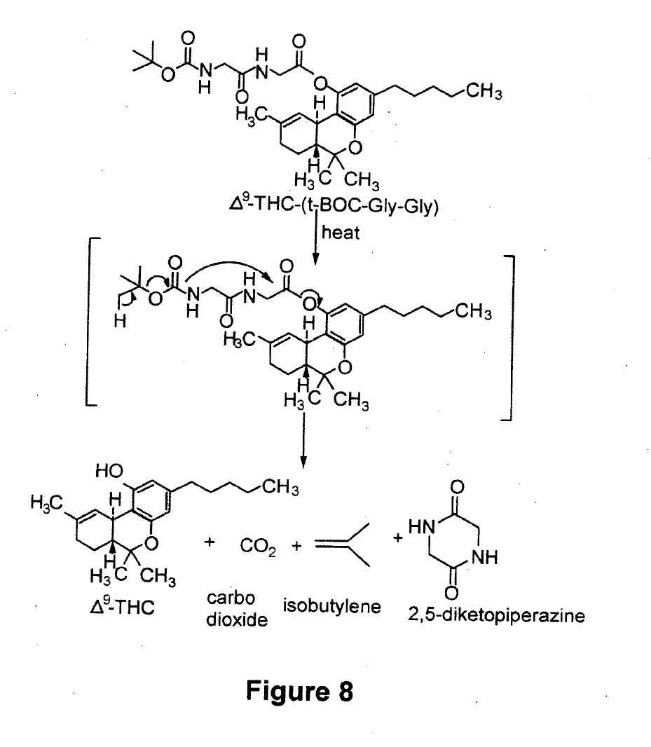

As shown in FIG. 8, an amino acid ester derivative of Δ⁹-THC (Δ⁹-THC-[1-(t-butoxycarbonyl-glycinyl-glycinate)] is thermally converted to Δ⁹-THC, with carbon dioxide, isobutylene and 2,5-diketopiperazine as by-products. 2,5-diketopiperazine (C₄H₆N₂O₂), a cyclic dimer of the amino acid glycine, which sublimes at 260° C. The general scheme for thermal conversion of a t-butoxycarbonyl-glycinyl-glycinate derivative of a drug is shown in FIG. 4.

### II. Propofol Prodrugs

The thermal conversion reactions described may be applied to drugs containing a phenol, such as, for example and without limitation, propofol, estradiol, apomorphine, dopamine, and epinephrine.

Propofol is a short-acting anaesthetic agent used for the induction of general anaesthesia in adult patients and pediatric patients older than 3 years of age; maintenance of general anesthesia in adult patients and pediatric patients older than 2 months of age; and sedation in medical context, such as intensive care unit (ICU) sedation for intubated, mechanically ventilated adults, and in invasive diagnostic procedures such as colonoscopy. Propofol is a water-immiscible oil that is typically administered intravenously as an emulsion of propofol in soybean oil and water.

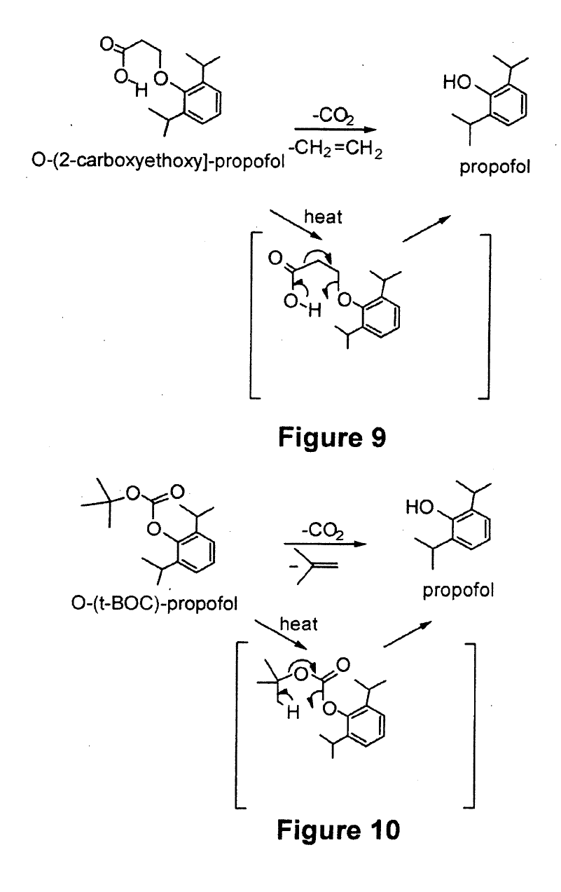

As shown in FIG. 9, the 0-2-carboxyethyl derivative of propofol (O-[2-carboxyethyl]-propofol) is thermally converted to propofol via a reverse Michael addition-type reaction, with carbon dioxide and ethylene as by-products.

As shown in FIG. 10, a t-butoxycarbonyl derivative of propofol (O-[t-butoxycarbonyl]-propofol) is thermally converted to propofol, with carbon dioxide and isobutylene as by-products.

### III. Estradiol Prodrugs

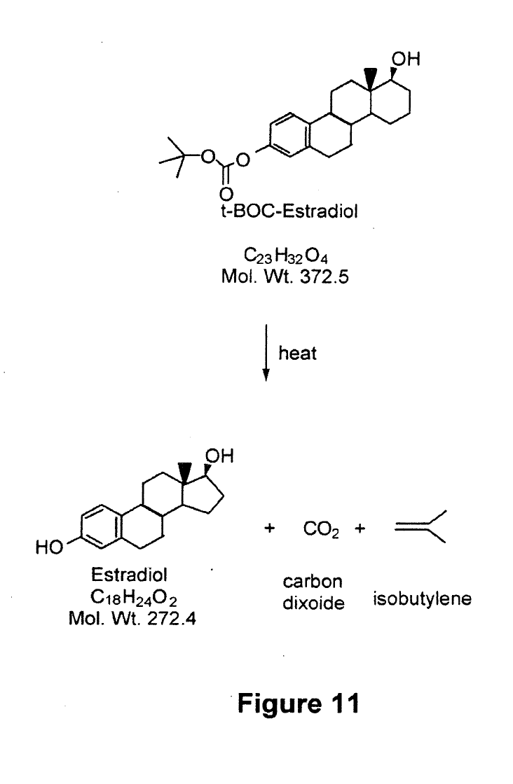

Estradiol is a derivative of cholesterol that represents the major estrogen in humans. Although primarily identified as a female hormone, estradiol is present to a lesser extent in males. Estradiol has not only a significant impact on reproductive and sexual functioning, but also affects other organs, including bone structure. Estradiol is most often prescribed for use in hormone replacement therapy for menopausal women. Estradiol is available in oral, transdermal, topical, injectable, and vaginal preparations. As shown in FIG. 11, a t-butoxycarbonyl derivative of estradiol (3-t-butoxycarbonyl-estradiol) is thermally converted to estradiol, with carbon dioxide and isobutylene as by-products.

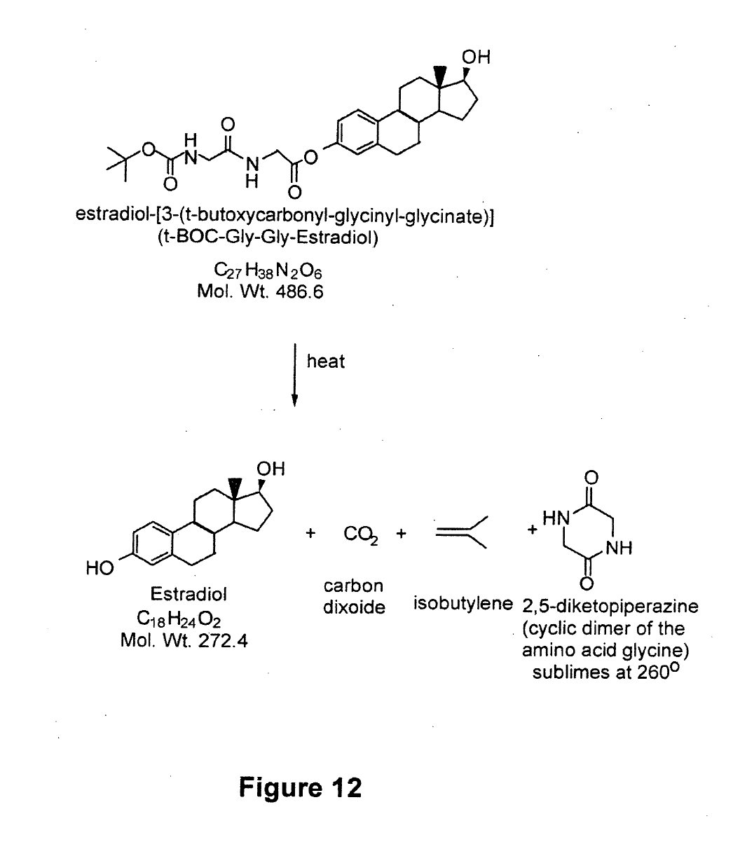

As shown in FIG. 12, a 3-(t-butoxycarbonyl-glycinyl-glycinate) derivative of estradiol is thermally converted to estradiol, with carbon dioxide, isobutylene and 2,5-diketopiperazine as by-products.

Empirical studies using model compound 3-(t-butoxycarbonyl-glycinyl-glycinate)]-estradiol gave up to 95% thermal conversion of the precursor to estradiol.

The following examples are presented to illustrate the present invention. It should be understood that the invention should not to be limited to the specific conditions or details described in these examples.

### EXAMPLES

Unless indicated otherwise, temperature is in degrees Celsius, and pressure is at or near atmospheric.

### Example 1

### Preparation of Δ⁹-THC-t-BOC-Gly-Gly

One gram (1 g; 3.2 mmole) of Δ⁹-THC was dissolved in 10 mL of DMF. DIEA (0.6 mL; 3.2 mmole) was added, followed by addition of 740 mg of N-(t-butyloxycarbonyl)-glycinylglycine (BOC-Gly-Gly; 3.2 mmole) and 1-ethyl-3-(3-dimethylaminopropyl)carbodiimide hydrochloride (EDAC, 610 mg, 3.2 mmole).

After 48 hours stirring at room temperature, the reaction was only about 50% complete, so an additional 740 mg of BOC-Gly-Gly (3.2 mmole) was added as a premixed solution in 5 mL of DMF containing 432 mg of hydroxybenzotriazole (HOBT; 3.2 mmole), 1.2 g of 2-(7-Aza-1H-benzotriazole-1-yl)-1,1,3,3-tetramethyluronium hexafluoro-phosphate (HATU; 3.2 mmole), and 1.7 mL of diisopropylethylamine (DIEA; 9.6 mmole). After stirring overnight at room temperature, the reaction mixture was diluted with ethyl acetate. The solution was washed with water, 10% aqueous citric acid, saturated aqueous sodium bicarbonate, and water, then dried over sodium sulfate, filtered, and evaporated. The residue was purified by column chromatography on silica gel using ethyl acetate/dichloromethane (20:80) as eluent. Yield was 910 mg of the Δ⁹-THC-t-BOC-Gly-Gly prodrug.

### Example 2

### Preparation of Propofol-T-BOC-Ester

Propofol (1.78 g; 10 mmole; obtained from Sigma-Aldrich, St. Louis, Mo.) was dissolved in 10 mL of tetrahydrofuran (THF). Dimethylaminopyridine (DMAP; 1.2 g; 10 mmole) was added to the propofol solution in an ice/methanol bath at −5° C., followed by dropwise addition of 2.18 g of t-butoxycarbonic acid anhydride (10 mmole). The ice/methanol bath was then removed and, after 3 hours stirring at room temperature, the reaction was complete. Work-up followed by silica gel chromatography using hexane/dichloromethane (50:50) provided a yield of 2.3 g of propofol-t-BOC ester prodrug.

### Example 3

### Preparation of T-BOC-Gly-Gly-Estradiol

Estradiol (1 g; 3.7 mmole; obtained from Sigma-Aldrich, St. Louis, Mo.) was dissolved in 10 mL of dimethylformamide (DMF). A premixed solution containing 944 mg of N-(t-butyloxycarbonyl)-glycinylglycine (BOC-Gly-Gly; 4 mmole), 1.5 g of 2-(7-Aza-1H-benzotriazole-1-yl)-1,1,3,3-tetramethyluronium hexafluorophosphate (HATU; 4 mmole), 540 mg of hydroxybenzotriazole (HOBT; 4 mmole), and 2.1 mL of diisopropylethylamine (DIEA; 12 mmole) in 10 mL of DMF was added to the estradiol solution.

The reaction mixture was stirred at room temperature for 24 hours, then poured into water and extracted with ethyl acetate. The organic layer was washed sequentially with 10% aqueous citric acid, saturated aqueous sodium bicarbonate, and water, then dried over sodium sulfate, filtered, and evaporated. The residue was purified by column chromatography on silica gel using ethyl acetate/dichloromethane (70:30) as eluent. Yield was 1.1 g of estradiol-t-BOC-Gly-Gly prodrug.

### Example 4

### Preparation of 1-(t-Butoxycarbonyl)-Δ⁹-THC

Δ⁹-THC (1.0 g; 3.2 mmole) was dissolved in 3 mL of dichloromethane (DCM). Dimethylaminopyridine (DMAP; 0.5 g; 3.8 mmole) was added to the THC solution in an ice/methanol bath at −5° C., followed by dropwise addition of 0.83 g of t-butoxycarbonic acid anhydride (3.8 mmole) in THF (3 mL). The ice/methanol bath was then removed and, after 3 hours stirring at room temperature, the reaction was complete. Work-up involved filtration through a plug of silica gel to give a quantitative yield of 1.32 g of t-BOC-Δ⁹-THC prodrug.

### Example 5

### Preparation of 1-(2-Carboxyethyl)-Δ⁹-THC

Δ⁹-THC (625 mg; 2 mmole) is added to a solution of potassium hydroxide 112 mg (2 mmole) in water (5 ml). The resulting mixture is heated to 50° C., then 3-bromopropionic acid (330 mg, 2.2 mmole) in water (5 ml) and potassium hydroxide 56 mg (1 mmole) in water (5 mL) are added alternately in small portions with stirring over 0.5 hour. The mixture is then cooled to room temperature, acidified with hydrochloric acid, and extracted with ether. The ether solution is filtered through a small plug of silica gel and evaporated to give 96 mg (25% calculated yield) of 1-(2-carboxyethyl) Δ⁹-THC.

### Example 6

### Preparation of 1-(2-Carboxyethyl)-Propofol

Propofol (1.78 g; 10 mmole) is added to a solution of potassium hydroxide (560 mg; 10 mmole) in water (10 mL). The resulting mixture is heated to 70° C., then 3-bromopropionic acid (1.53 g; 10 mmole) in water (10 mL) and potassium hydroxide (280 mg; 5 mmole) in water (5 mL) are added alternately in small portions with stirring over 0.5 hours. The mixture is refluxed for 10 minutes and then cooled to room temperature and washed with ether. The aqueous solution is acidified with hydrochloric acid and extracted with ether. The ether solution is filtered through a small plug of silica gel and evaporated to give 1.25 g (50% calculated yield) of 1-(2-carboxyethyl)-propofol.

### Example 7

### Preparation of 3-(t-Butoxycarbonyl)-Estradiol

Estradiol (544 mg; 2 mmole) is dissolved in 3 mL of dichloromethane (DCM). Dimethylaminopyridine (DMAP; 0.32 g; 2.4 mmole) is added to the estradiol solution in an ice/methanol bath at −5° C., followed by dropwise addition of 0.52 g of t-butoxycarbonic acid anhydride (2.4 mmole) in THF (3 mL). The ice/methanol bath is then removed and, after 3 hours stirring at room temperature, the reaction is complete. Work-up involves filtration through a plug of silica gel to give a 0.74 g (100% calculated yield) of 3-(t-butoxycarbonyl)-estradiol prodrug.

### Example 8

### Δ⁹-THC-2-Carboxylic Acid (THCA) Coated Films

Coated substrates were generated by spray depositing prodrug, Δ⁹-THC-2-carboxylic acid, solution (about 50 mg/mL prodrug in organic solvent) onto a small section of a laser-cut stainless steel (SS) foil coupon (SAE 304, 1=6 cm, w=1.25 cm, t=0.01 cm). The spray coating system consisted of an ultrasonic nozzle spray nozzle (Sono-Tek Corp, Milton, N.Y.) mounted on a Cartesian robot and fed by a calibrated syringe pump. The prodrug loading (coated mass normalized over coated surface area [mg/cm2]) was accurately controlled by varying the coating surface area and the syringe pump delivery rate. The coat content and prodrug loading were verified by recovering the coated prodrug from the foil in organic solvent and analyzing the solution using high performance liquid chromatography (HPLC). The solvent was evaporated, leaving behind a prodrug film.

A 6-month comprehensive stability study of Δ⁹-THC-2-carboxylic acid (THCA; shown in FIG. 3; obtained from Aphios Corporation, Woburn, Mass.) coated onto vaporization substrates (1 mg/cm²) was conducted using three temperature and relative humidity (RH) conditions:

1) 25° C.+60% RH (normal room conditions);

2) 40° C.+75% RH (FDA accelerated stability conditions); and

3) 40° C.+anhydrous.

By the end of 6 months, the samples stored at normal room conditions experienced an inconsequential loss in purity, whereas the samples stored at 40° C. and 75% RH experienced a nearly 60% loss in chemical purity.

The major degradant identified in the accelerated stability condition was the therapeutic Δ⁹-THC. This is an acceptable degradant. When stored at normal room temperature and humidity, the chemical integrity of THCA coated on stainless steel foil is preserved for at least 6 months.

Previous studies indicated that dronabinol (THC) remains only 60% pure after 4 weeks dark storage at room temperature and humidity, and roughly 30% pure after 14 weeks in the same conditions. Hence, in comparison with pure THC, THCA presents a viable formulation strategy for addressing this shelf-life issue. The major known degradation products comprised >0.5% total peak area (confirmed with internal standards). The corresponding fraction of total peak area for 1.0 mg/cm² THCA coatings stored at 40° C. and 75% relative humidity (worst case scenario) are set forth in Table One, below.

TABLE 1
Major Degradation Products Identified After a
6-Month Stability Study of THCA Coated onto SS
Foils and Stored at 40° C. and 75% RH

| | | | | | |
|---|---|---|---|---|---|
| HPLC | 0.76 | 0.82 | 0.85 | 0.96 | 1.1 |
| RRT * | | | | | |
| Degradant | Cannabinol | Δ⁹-THC | Δ⁸-THC | Cannabinolic | Δ⁸-THCA |
| Name | | | | Acid | |
| % Total | 8.5 | 12.9 | 2.6 | 10.8 | 1.2 |
| Peak Area | | | | | |

* RRT = Relative retention time to THCA (RT = 34.1 min); 60 min gradient with acidic mobile phase; detection at 215 nm.

An advantage of THCA is that it is a solid that forms a physically stable film, as opposed to Δ⁹-THC, which is a viscous oil whose coated films are subject to flow. Physical stability drop tests indicated that THCA coatings (maximum loading tested was 1.0 mg/cm²) on stainless steel substrates are physically robust, even after 6 months storage at various environmental conditions.

### Example 9

Δ⁹-THC-2-Carboxylic Acid (THCA) Vaporization and Aerosol Generation

Aerosols were generated using a bench-top screening device operated by discharging a capacitor in circuit with the drug-coated foil. Electrical resistance rapidly (within <500 msec) heats the drug-coated foil to a selectable vaporization temperature. Thermophoresis draws the drug vapor away from the foil, while air drawn across the foil from an in-house vacuum facilitates the recondensation of the vapor to form drug aerosol particles.

The aerosol was collected with either a Teflon filter for quality analysis or using an Anderson-type Cascade Impactor (ACI) for particle sizing. The aerosol was extracted from the collection apparatus using organic solvent and was analyzed using HPLC.

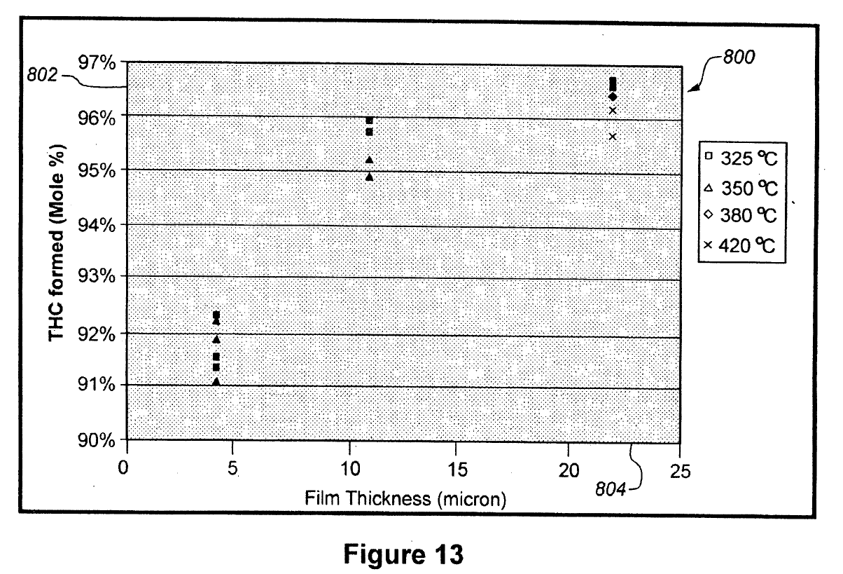

Initial vaporization tests of THCA coated onto stainless steel foils indicated that prodrug conversion is proportional to drug loading (linear fit R²⁼⁸⁵%), with an asymptote appearing around 92% (drug loading≈1.6 mg/cm²). FIG. 13 is a plot 1300 showing THC formed (mole %) 1302 as a function of coated film thickness 1304.

As shown in FIG. 13, films with higher drug loadings (i.e., film thicknesses) had higher prodrug conversion rates than films with lower drug loadings. This was attributed to the fact that heat transfer mechanisms in thicker films increase the temporal duration that the drug is exposed to decarboxylation conditions, relative to that of thinner films. In essence, the thicker the drug film, the longer the drug is heated. The decarboxylation kinetics were optimized for our bench-top vaporization apparatus using drug loadings in the 1 mg/cm² range and a vaporization temperature in the range of 350° C. to 375° C.

Table Two, below, summarizes the results from a conversion optimized vaporization experiment of THCA test articles heated to 368° C. using the electrical bench-top apparatus.

TABLE 2
Summary of the Results of an Optimized
Vaporization Study of THCA (n = 5)

| | Quality Parameter | Mean (RSD) |
|---|---|---|
| Coating | Coated Dose (THCA + THC) | 1101.8 μg (2%) |
| | Coated Drug Composition (% THCA) | 98% |
| | Drug Loading | 1.3 mg/cm² |
| Aerosol | Vaporization Temperature | 368° C. |
| | THCA → THC Conversion Efficiency (molar) | 91.4% (0.6%) |
| | Emitted Dose THC | 913.2 μg (3%) |
| | Aerosol Yield (THC + THCA) | 101% (3%) |

As shown in Table 2, above, both the coating and vaporization processes were highly reproducible, with relative standard deviations (RSD) of less than 5%. In addition, the aerosol comprised over 90% THC, indicating a relatively efficient conversion process. Efforts to improve the conversion efficiency (device modifications allowing slower heating, step-wise heating, and/or improving coating height uniformity) increased the conversion efficiency to about 94%.

The aerodynamic diameter of an aerosol particle is one of the key defining properties that dictate pulmonary deposition and absorption. Particles with aerodynamic diameters larger than 5 μm risk deposition in the throat or upper airway, while particles with aerodynamic diameters smaller than 1 μm may be exhaled before having a chance to settle in the deep lung. These guidelines are strongly dependent on individual breathing habits such as breath-hold; nevertheless, the particle size distribution is currently used in the pharmaceutical industry as a predictor of the efficacy of deep lung drug delivery.

Particle size distribution is characterized by the mass median aerodynamic diameter (MMAD) and the geometric standard deviation (GSD). For thermal condensation aerosols, such as those disclosed herein and in our previous patent applications, particle size is governed by the competing mechanisms of condensation and Brownian aggregation. The density of drug vapor in the airflow, and hence air flow rate and drug mass, governs the condensation/aggregation kinetics.

All particle size experiments were conducted by vaporizing drug films into an 8-stage Anderson Cascade Impactor (ACI) fitted with a glass fiber filter. The ACI consists of several stages, with each successive stage having a smaller size cutoff. By extracting and determining the mass of drug deposited at each stage, it is possible to estimate the particle size distribution of the aerosol.

An air flow of 28.3 L/min was used to generate the aerosol and distribute it through the ACI. Each stage and filter was extracted with organic solvent and analyzed using HPLC. The MMAD and GSD were calculated from the quantity of aerosol on each stage. The MMAD for the THC aerosol (generated from THCA film, drug loading=1 mg/cm²; aerosol mass=1 mg) was 2.2 μm and the GSD [84/16] was 2.2. In addition, the fine particle fraction (FPF, MMAD <5 μm) was over 95%. These values are well within the range normally accepted for effective pulmonary deposition.

### Example 10

### Delivery of Aerosolized THC

A study was conducted in order to compare the pharmacokinetics (PK) of delivering THC by inhalation from a thermally labile prodrug to those of an intravenous (IV) bolus. A device consisting of a control electronics PC board, air-flow regulator, inhalation valve, air-flow meter, and indo-tracheal tube was used to generate and administer THC aerosol to Beagle dogs. Two safety mechanisms built into the device prevent harm to the test subject: one that closes the inhalation valve when the selected air volume is delivered to the test subject, and one that vents the system to ambient air if the circuit board loses control over the system (due to power failure, etc.).

The in vivo portion of the study was designed with a target THC emitted dose of 0.98 mg. Aerosol quality samples were captured prior to and immediately following animal dosing in a manner consistent with previous pre-PK development work and the animal dosing parameters. The emitted dose samples were collected on 2 μm Teflon filters, while the particle size samples were collected using an ACI fitted with glass fiber filters. After aerosol collection, the filters were stored in amber vials in a freezer prior to analysis. All results were within the acceptable range determined from a previous development study.

For the inhalation portion, four Beagle dogs (2 male/2 female) were exposed to THC aerosol by forced maneuver inhalation exposure. A breath hold of 5 sec was followed by exhalation into a Teflon filter. The filters were placed in vials for analysis using HPLC. Body weight, inhalation volume, exhalation volume, and quantity of THC in exhalation for each test subject are set forth in Table Three, below.

TABLE 3
Biological and Pulmonary Parameters of In Vivo Test Subjects

| | | Weight Prior | Weight Prior | | Fraction of | THC | Fraction of THC |
|---|---|---|---|---|---|---|---|
| | | to Inhalation | to IV | Exhalation | Intake Air | in Exhaled | Emitted Dose in |
| Subject | Sex | Dosing (kg) | Dosing (kg) | Volume (L) | Exhaled (%) | Breath (μg) | Exhalation (%) * |
| 101 | M | 9.10 | 9.57 | 0.696 | 106 | 26.2 | 2.8 |
| 102 | M | 10.50 | 10.89 | 0.434 | 66 | 29.6 | 3.2 |
| 103 | F | 10.23 | 10.25 | 0.421 | 74 | 22.3 | 2.4 |
| 104 | F | 8.10 | 8.05 | 0.412 | 68 | 15.8 | 1.7 |

* Based on 924 μg THC emitted dose (average of pre- and post-dose aerosol quality tests).

Two weeks after dosing by inhalation, the same dogs were given a 0.9 mL (adjusted to match the aerosol output measured in the pre- and post-dose aerosol quality testing) bolus IV injection of THC (1 mg/mL THC and sodium ascorbate) in 0.9% NaCl aqueous solution.

For both inhalation and injection routes, arterial blood was sampled from an in-dwelling catheter in the ascending aorta at various time points over the pre-dose period to 10 minutes post-dose, while venous blood was sampled from an in-dwelling catheter in the superior vena cava at various time points pre-dose to 24 hours post-dose. All blood samples measured 0.5 mL and were collected in plastic vials containing K₂EDTA as the anticoagulant. The vials were placed on wet ice until centrifugation to recover the plasma. The plasma samples were analyzed for THC, THCA, and the major metabolite (11-nor-9-carboxy-Δ⁹-tetrahydrocannabinol) of THC using a mass spectrometry method.

Immediately after simultaneous administration of the drug and the prodrug, blood samples were drawn from both the arterial and venous circulation of the test subjects. Arterial sampling was more frequent at the early time points and was stopped at 10 minutes, while venous sampling continued to 24 hours.

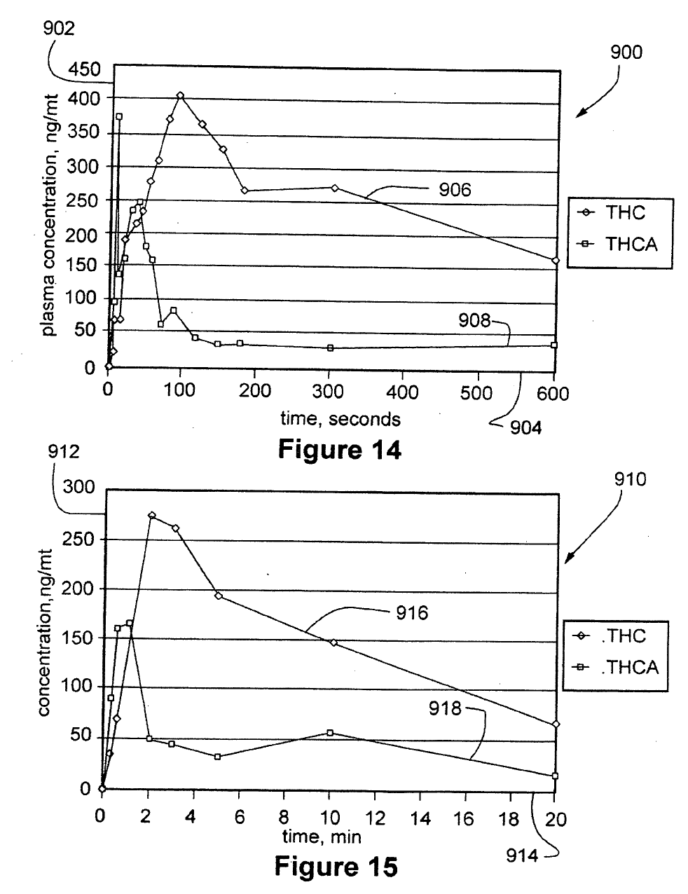

FIG. 14 is a plot 1400 showing arterial plasma concentration 1402 of THC 1406 and THCA 1408 as a function of time 1404. FIG. 15 is a plot 1500 showing venous plasma concentration 1502 of THC 1506 and THCA 1508 as a function of time 1504.

The data shown in FIGS. 14 and 15 clearly indicate that the THCA prodrug is absorbed from the lung substantially faster than the THC. The arterial concentration data show a maximum concentration of the prodrug occurring at about 30 seconds, while the drug concentration peaks at 90 seconds. The venous concentration data show the prodrug peak at about 50 seconds and the drug peak at about 150 seconds.

In summary, delivery of aerosolized THC to dogs via inhalation resulted in rapid systemic absorption and high bioavailability of THC, with demonstrable and significant differences (3-fold) in time to maximum plasma concentration (Tₘₐₓ) between THC and its prodrug Δ⁹-THC-2-carboxylic acid. These differences may be attributable to differences in the physicochemical properties of the two molecules.

Non-compartmental modeling of the venous THC plasma concentrations was performed using WinNonlin™ (WinNonlin Professional Version 4.1, Pharsight Corp., Mountain View, Calif.). Characterization of the gamma (third) terminal linear phase for the inhalation and IV portions of the study allowed us to compare the inhalation pharmacokinetics to those of IV.

The parameters of the analysis are defined as follows:

Cₘₐₓ Maximum (peak) plasma concentration

Tₘₐₓ Time of maximum (peak) plasma concentration

AUC∞ Area under the concentration-time curve, extrapolated to infinity using the log-linear regression analysis of the concentration-time data in the terminal phase.

$$B A ( Bioavailability ) : \frac{A U {C}_{∞}^{inhalation} \cdot {dose}^{inhalation}}{A U {C}_{∞}^{IV} \cdot {dose}^{IV}}$$

Results of the in vivo pharmacokinetic (PK) study are summarized in Tables Four and Five, below.

TABLE 4
Summary of the In Vivo Pharmacokinetic Results: Administration
by IV Injection (Dose = 0.9 mg THC)

| PK | | Subject | | | | | | RSD |
|---|---|---|---|---|---|---|---|---|
| Parameter | Unit | 101 | 102 | 103 | 104 | Mean | SD | (%) |
| Half-life | Min | 18.1 | 97.1 | 51.3 | 36.6 | 50.8 | 33.75 | 66.5 |
| Tₘₐₓ | Min | 0.25 | 0.5 | 0.25 | 0.5 | 0.38 | 0.14 | 36.8 |
| Cₘₐₓ | ng/mL | 3772 | 697 | 1406 | 1234 | 1777 | 1364 | 76.7 |
| AUC∞ | min* ng/mL | 9821 | 8780 | 8313 | 7088 | 8500 | 1133 | 13.3 |

*Limit of quantification (LOQ) = 1 ng/mL.

TABLE FIVE
Summary of the In Vivo Pharmacokinetic Results: Administration
by Inhalation (Dose = 0.93 mg THC + 0.11 THCA)

| PK | | Subject | | | | | | RSD |
|---|---|---|---|---|---|---|---|---|
| Parameter | Unit | 101 | 102 | 103 | 104 | Mean | SD | (%) |
| Half-life | Min | 21.0 | 32.5 | 22.7 | 55.8 | 33.0 | 16.0 | 48.5 |
| Tₘₐₓ | Min | 3.0 | 2.0 | 2.0 | 2.0 | 2.3 | 0.5 | 21.7 |
| Cₘₐₓ | ng/mL | 341 | 190 | 358 | 253 | 286 | 78.2 | 27.4 |
| AUC∞ | Min* ng/mL | 5963 | 3872 | 68156 | 5554 | 5551 | 1237 | 22.3 |
| BA | % | 59.7 | 43.9 | 84.5 | 81.6 | 67.4 | 19.2 | 28.5 |

*Limit of quantification (LOQ) = 1 ng/mL.

Mean THC bioavailability was 67% and peak plasma levels occurred in 2 to 3 minutes. In contrast, oral dronabinol formulations of THC typically have a bioavailability less than 50% and Tₘₐₓ can be up to 5 hours.

### Example 11

### Aerosolization of Propofol-t-BOC-Ester

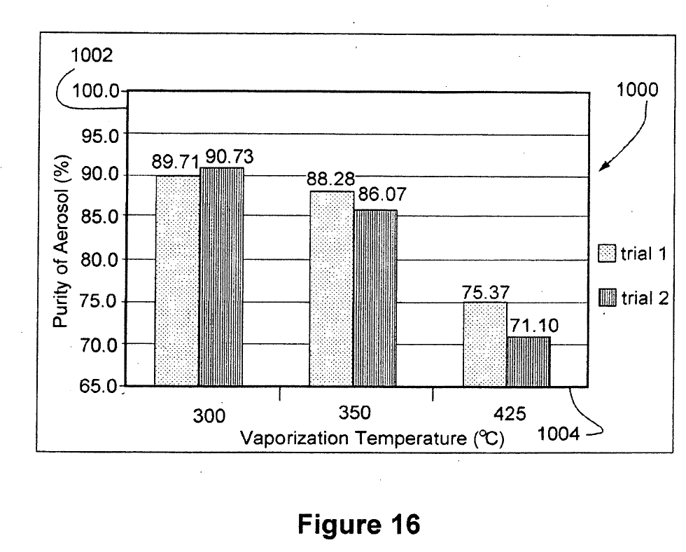

FIG. 16 is a bar graph 1600 showing aerosol purity 1602 of propofol as a function of vaporization temperature 1604 (n=2). Aerosol purity is shown to decrease with increasing vaporization temperature.

### Example 12

### Aerosolization of Estradiol-t-BOC-Gly-Gly

The prodrug, estradiol-t-BOC-Gly-Gly (shown in FIG. 12 and prepared as described above), was spray coated onto stainless steel test strips (1.347 mg; 2.41 Mm nominal film thickness). The test articles were placed in the screening device and heated by discharging a capacitor through the foils to thermally convert the prodrug back to estradiol and form a condensation aerosol. Tests were conducted on duplicate foils at each of three temperatures (325° C., 350° C., and 380° C.) determined by the discharge voltage of the capacitor. The aerosol was collected with a Teflon filter, and the trapped aerosol was extracted from the collection apparatus using organic solvent and analyzed using HPLC.

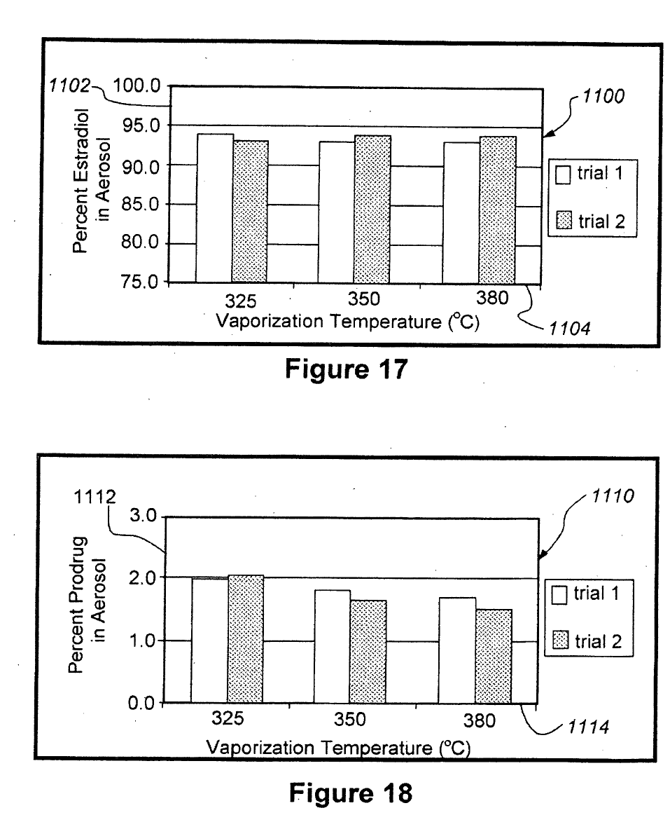

FIG. 17 is a bar graph 1700 showing percent estradiol in aerosol 1702 as a function of vaporization temperature 1704 (n=2). FIG. 18 is a bar graph 1800 showing percent prodrug in aerosol 1802 as a function of vaporization temperature 1804 (n=2).

As shown in FIGS. 17 and 18, the composition of the aerosol was approximately 94% estradiol; approximately 1.5-2% of the captured aerosol consisted of unconverted prodrug, along with minor amounts of side products.

It is to be understood that, while the invention has been described in conjunction with the preferred specific embodiments thereof, that the description above as well as the examples that follow are intended to illustrate and not limit the scope of the invention. The practice of the present invention will employ, unless otherwise indicated, conventional techniques of chemistry, manufacturing and engineering, and the like, which are within the skill of the art. Other aspects, advantages and modifications within the scope of the invention will be apparent to those skilled in the art to which the invention pertains. Throughout the specification, any and all references to a publicly available document, including but not limited to a U.S. patent, are specifically incorporated by reference.

What is claimed is:

1. A prodrug of a phenolic drug compound, the prodrug having the general structural formula:
DRUG-O—(CR¹R²)ₙCOOR³
wherein
DRUG-O— is a hydroxyl functional group attached to a carbon atom of an aromatic ring of the phenolic drug compound;
R¹, R² and R³ are independently selected from the group consisting of H, cycloalkyl groups having up to 10 carbon atoms, straight or branched chain alkyl, alkenyl or alkynyl groups of 1 to 10 carbon atoms, wherein the chains thereof (i) may be interrupted by at least one N, S, or O atom, or (ii) may be substituted by at least one group selected from the group consisting of COR⁴, COOR⁴ and CON(R⁴)₂, hydrocarbyl aryl groups, aryl groups substituted by at least one group selected from the group consisting of COR⁴, COOR⁴, CON(R⁴)₂, N(R⁴)₂, OR⁴, halogen, SR⁴, NO₂, and R⁴, mono- bi-cyclic saturated or unsaturated heterocyclic rings, each ring consisting of 3 to 7 members selected from the group consisting of carbon, nitrogen, oxygen and sulfur, CN, COR⁴, COOR⁴, CON(R⁴)₂, and C(halogens)₃;
R⁴ is selected from the group consisting of cycloalkyl groups having up to 10 carbon atoms, straight or branched chain alkyl, alkenyl and alkynyl groups having 1 to 10 carbon atoms, straight or branched chain alkyl, alkenyl and alkynyl groups of 1 to 10 carbon atoms wherein the chains thereof may be interrupted by at least on N, S or O atom, hydrocarbyl arl groups, and in the case of —N(R⁴)₂ taken with the other R⁴ group and N is mono- or bi-cyclic saturated or unsaturated heterocyclic ring, wherein each ring consists of 3 to 7 members selected from the group consisting of carbon, nitrogen, oxygen and sulfur;
n is 1 to 3; and
(B) salts thereof.

2. The prodrug of claim 1, wherein the phenolic drug compound is selected from the group consisting of Δ⁹-tetrahydrocannabinol, propofol, and estradiol.

3. The prodrug of claim 2, wherein R¹, R² and R³ are H, and n is 2.

4. A prodrug of a phenolic drug compound, the prodrug having the general structural formula: 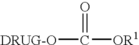
wherein
DRUG-O— is a hydroxyl functional group attached to a carbon atom of an aromatic ring of the phenolic drug compound;
R¹ is selected from the group consisting of H, cycloalkyl groups having up to 10 carbon atoms, straight or branched chain alkyl, alkenyl or alkynyl groups of 1 to 10 carbon atoms, wherein the chains thereof (i) may be interrupted by at least one N, S, or O atom, or (ii) may be substituted by at least one group selected from the group consisting of COR², COOR² and CON(R²)₂, hydrocarbyl aryl groups, aryl groups substituted by at least one group selected from the group consisting of COR², COOR², CON(R²)₂, N(R²)₂, OR², halogen, SR², NO₂, and R², mono- bi-cyclic saturated or unsaturated heterocyclic rings, each ring consisting of 3 to 7 members selected from the group consisting of carbon, nitrogen, oxygen and sulfur, CN, COR², COOR², CON(R²)₂, and C(halogens)₃;
R² is selected from the group consisting of cycloalkyl groups having up to 10 carbon atoms, straight or branched chain alkyl, alkenyl and alkynyl groups having 1 to 10 carbon atoms, straight or branched chain alkyl, alkenyl and alkynyl groups of 1 to 10 carbon atoms wherein the chains thereof may be interrupted by at least on N, S or O atom, hydrocarbyl arl groups, and in the case of —N(R²)₂ taken with the other R² group and N is mono- or bi-cyclic saturated or unsaturated heterocyclic ring, wherein each ring consists of 3 to 7 members selected from the group consisting of carbon, nitrogen, oxygen and sulfur; and
(B) salts thereof.

5. The prodrug of claim 4, wherein the phenolic drug compound is selected from the group consisting of Δ⁹-tetrahydrocannabinol, propofol, and estradiol.

6. The prodrug of claim 5, wherein R¹ is H.

7. A method of making a phenolic drug compound comprising heating a composition comprising a prodrug of the phenolic drug compound to a temperature greater than 100°, wherein the prodrug is a prodrug of claim 4.

8. A method of making a vapor comprising a phenolic drug compound comprising heating a composition comprising a prodrug of the phenolic drug composition to a temperature sufficient to vaporize at least a portion of the composition to generate a vapor comprising the phenolic drug compound.

9. The method of claim 8 further comprising condensing the vapor to form an aerosol.

10. A prodrug of a phenolic drug compound, the prodrug having the general structural formula: 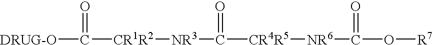
wherein
DRUG-O— is a hydroxyl functional group attached to a carbon atom of an aromatic ring of the phenolic drug compound;
R¹, R², R³, R⁴, R⁵, R⁶ and R⁷ are independently selected from the group consisting of H, cycloalkyl groups having up to 10 carbon atoms, straight or branched chain alkyl, alkenyl or alkynyl groups of 1 to 10 carbon atoms, wherein the chains thereof (i) may be interrupted by at least one N, S, or O atom, or (ii) may be substituted by at least one group selected from the group consisting of COR⁸, COOR⁸ and CON(R⁸)₂, hydrocarbyl aryl groups, aryl groups substituted by at least one group selected from the group consisting of COR⁸, COOR⁸, CON(R⁸)₂, N(R⁸)₂, OR⁸, halogen, SR⁸, NO₂, and R⁸, mono-bi-cyclic saturated or unsaturated heterocyclic rings, each ring consisting of 3 to 7 members selected from the group consisting of carbon, nitrogen, oxygen and sulfur, CN, COR⁸, COOR⁸, CON(R⁸)₂, and C(halogens)₃;
R⁸ is selected from the group consisting of cycloalkyl groups having up to 10 carbon atoms, straight or branched chain alkyl, alkenyl and alkynyl groups having 1 to 10 carbon atoms, straight or branched chain alkyl, alkenyl and alkynyl groups of 1 to 10 carbon atoms wherein the chains thereof may be interrupted by at least on N, S or O atom, hydrocarbyl arl groups, and in the case of —N(R⁸)₂ taken with the other R⁸ group and N is mono- or bi-cyclic saturated or unsaturated heterocyclic ring, wherein each ring consists of 3 to 7 members selected from the group consisting of carbon, nitrogen, oxygen and sulfur; and
(B) salts thereof.

11. The prodrug of claim 10, wherein the phenolic drug compound is selected from the group consisting of Δ⁹-tetrahydrocannabinol, propofol, and estradiol.

12. The prodrug of claim 11, wherein R¹, R², R³, R⁴, R⁵, R⁶ and R⁷ are H.

13. A method of making a phenolic drug compound comprising heating a composition comprising a prodrug of the phenolic drug compound to a temperature greater than 100°, wherein the prodrug is a prodrug of claim 10.

14. A method of making a vapor comprising a phenolic drug compound comprising heating a composition comprising a prodrug of the phenolic drug composition to a temperature sufficient to vaporize at least a portion of the composition to generate a vapor comprising the phenolic drug compound.

15. The method of claim 14 further comprising condensing the vapor to form an aerosol.

16. A prodrug of a phenolic drug compound, the prodrug having the general structural formula: 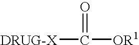
wherein
DRUG-X— is a carbon atom of an aromatic ring of the phenolic drug compound in the o- or p- position relative to a hydroxyl functional group attached to a different carbon atom of said aromatic ring;
R¹ selected from the group consisting of H, cycloalkyl groups having up to 10 carbon atoms, straight or branched chain alkyl groups of 1 to 10 carbon atoms; and
(B) salts thereof.

17. The prodrug of claim 16, wherein the phenolic drug compound is selected from the group consisting of Δ⁹-tetrahydrocannabinol, propofol, and estradiol.

18. The prodrug of claim 17, wherein R¹ is H.

19. A method of making a phenolic drug compound comprising heating a composition comprising a prodrug of the phenolic drug compound to a temperature greater than 100°, wherein the prodrug is a prodrug of claim 16.

20. A method of making a vapor comprising a phenolic drug compound comprising heating a composition comprising a prodrug of the phenolic drug composition to a temperature sufficient to vaporize at least a portion of the composition to generate a vapor comprising the phenolic drug compound.

21. The method of claim 20 further comprising condensing the vapor to form an aerosol.
